(19) United States
(12) Patent Application Publication
(10) Pub. No.: US 2007/0104610 A1
(43) Pub. Date: May 10, 2007

(54) Plasma sterilization system having improved plasma generator

(76) Inventors: Edward J. Houston, East Brunswick, NJ (US); Christopher S. Brockman, Kalamazoo, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/591,363

(22) Filed: Nov. 1, 2006

Related U.S. Application Data
(60) Provisional application No. 60/732,616, filed on Nov. 1, 2005.

(51) Int. Cl. A61L 2/14; H05B 7/00

(52) U.S. Cl. 422/22; 422/28; 373/18

(57) ABSTRACT

A plasma sterilization system employing an improved plasma generator exhibiting low pressure drop of fluid passing through the plasma generator, and/or a homogeneous distribution of current through the space between a primary planar electrode and a secondary planar electrode in accordance with certain preferred embodiments includes at least one dielectric material having a tortuous porous structure disposed adjacent at least one of the electrodes, and a plasma generating chamber defining an enclosure having a fluid inlet and a fluid outlet, in which the inlet and outlet are located laterally of and between planes coinciding with major surfaces of the planar electrodes.

### CROSS-REFERENCE TO RELATED APPLICATION

This application claims priority under 35 U.S.C. § 119(e) to U.S. Provisional Application No. 60/732,616 entitled POROUS ELECTRODES FOR USE WITH PLASMA REACTORS AND METHOD FOR USING THE SAME, filed Nov. 1, 2005, by Edward J. Houston Jr., the entire disclosure of which is incorporated herein by reference.

### FIELD OF THE INVENTION

This invention relates to a plasma emitting apparatus (plasma generator), and the use of plasma emitting apparatus for the sterilization of articles, such as medical instruments, and more particularly to plasma emitting apparatus employing a porous dielectric disposed on a face of at least one electrode and to a process for sterilizing an article by contacting the article with a fluid discharged from the plasma emitting apparatus.

### BACKGROUND OF THE INVENTION

A plasma is a partially ionized gas produced by high temperature (such as in a flame) or by a strong electric field, which may be generated either by a direct current (DC) or by a time varying current (typically at a radio wave or microwave frequency). A plasma may comprise, in addition to ions, photons, metastable species, species having an atomic excited state, free radicals, molecular fragments and electrons, depending on the composition of the gas being ionized and on the operating parameters of the plasma generator. The chemical species in a plasma are chemically active, and may be used to modify surfaces, including sterilization of surfaces.

Thermal plasmas produced by high temperature are not generally suitable for sterilizing non-heat resistant articles (i.e., heat-sensitive articles), such as plastic components that would decompose or degrade when exposed to high temperatures. Accordingly, plasmas used for sterilizing heat-sensitive articles are preferably generated using a strong electric field.

Plasma generators which produce ions in a strong electric field comprise a primary electrode, a secondary electrode spaced from the primary electrode, and a power source for establishing an electric potential and an electric field between the electrodes. A plasma is generated in a space generally defined between the electrodes.

While it is conceivable that a plasma generator operating in an arcing mode could be used to produce a plasma suitable for sterilizing surfaces of articles, glow discharge plasma generators are preferred for a variety of reasons, including improved plasma density (i.e., the number of electrons and ions produced per unit volume of gas between the electrodes), increased efficiency, reduced damage to electrodes, and lower temperature. In the glow discharge plasma generators, electrical current passing through the space between the electrodes is highly dispersed causing a glow between the electrodes rather than an arc. A stable glow discharge can be maintained by employing a high frequency (e.g., greater than about 1 kHz), by diluting the gas with helium (or other noble gas) or by employing brush-style or metal wire mesh electrodes. The apparatus needed for providing high frequency AC at the voltages desired for generating a plasma can be expensive. Likewise, diluent noble gases (such as helium, argon, neon, etc.) are expensive. Complicated electrode structures can undesirably reduce plasma density and efficiency.

A preferred technique for achieving a low-temperature, high density plasma involves the use of at least one porous dielectric material positioned between the electrodes. The individual pores of the porous dielectric material act as current-limiting micro channels which prevent a current density above the threshold for arc discharge between the electrodes. Instead, the current is highly dispersed causing a relatively homogeneous glow between the electrodes rather than an arc.

It has been proposed to use a non-thermal plasma generator to produce a continuous flow of low-temperature plasma at or near ambient pressure to a sterilization chamber containing articles, such as medical instruments, which are to be sterilized. The low pressures and temperatures of the plasma allow safe and effective sterilization of articles having plastic, elastomeric or other heat-sensitive components.

However, creating a non-thermal air plasma, known as a glow discharge, at atmospheric pressure can be difficult. As the gas in the discharge region heats up, the electrical conductivity of the air increases resulting in an increase in the discharge current. Soon after narrow filamentary discharges, typically only a few microns wide, known as streamers, begin to form. As more and more current passes through the streamer, the gas in the vicinity of the streamer continues to heat up eventually collapsing into a hot and destructive thermal plasma discharge, which is known as an arc. This process is known as the glow-to-arc transition.

One of the most useful features of glow discharges is its highly chemical active nature which extends over a large volume. As the glow plasma begins to collapse into the micron size streamers, the energy is confined to rather small, discrete regions of the original volume. These streamers tend to be inefficient in enhancing the overall chemistry of a system since the bulk of the gas is inaccessible to these localized streamers. When the plasma eventually collapses into an arc, the problems are actually magnified. In addition to the destructive nature of the arc, the plasma typically collapses into a single arc filament, which is very localized but the intense heat generated by the filament tends to change the whole chemistry of the system.

In recent years, several devices have been developed to suppress the glow-to-arc transition to create a “glow-like” plasma at atmospheric pressures. More specifically, capillary discharge devices, micro-hollow cathode discharge devices, slit discharge devices and slot discharge devices, etc., have been developed to suppress the glow-to-arc transition. However, all of these devices effectively accomplish this task under particular operating conditions, i.e., the slit discharge operates well at a power supply frequency of 60 Hz but not at 20 kHz, whereas, the capillary plasma operates better at a frequency of 20 kHz compared to the 60 Hz frequency.

Accordingly, none of the above configurations have demonstrated the ability to maintain a stable, diffuse glow plasma in air at atmospheric pressure. Instead, they operate by producing stable plasma jets or plumes, which, while far superior in enhancing the chemistry of a bulk gas, still suffer from localized intense plasma regions rather than maintaining a uniform diffuse glow. Furthermore, only the micro-hollow cathode discharge has been shown to operate in air using direct current and in that case, only two plumes spaced 4 mm apart were operated. In addition, that particular device required the use of ballast resistors to operate in atmospheric conditions.

It is therefore desirable to solve the aforementioned problems associated with destructive thermal discharges in the form of arcs.

While the gases entering the plasma generator in the apparatus described above could consist exclusively of ambient air, and would result in the production of ozone and possibly other species that could be effectively used for sterilization, faster and more effective sterilization can be achieved by adding an organic additive, such as an alcohol (e.g., C₁-C₅ alcohol) and/or an alkene (e.g., a C₂ -C₆ alkene), as disclosed in United States Patent Application Publication No. 2004/0050684, which is incorporated herein by reference in its entirety. It may also be desirable to add an oxidizer, such as oxygen.

The known plasma generators that have been proposed and/or employed for producing a stream of low-temperature (e.g., from ambient to about 50° C.) plasma have generally been designed so that the fluids (gases and/or plasmas) passing through the plasma generator flow through a segmented or perforated electrode, and typically make at least one right-angle turn before existing the plasma generator. This arrangement has the advantage of utilizing the same conduit for passage of fluid as an electrical conductor, and also may serve to cool the segmented or perforated electrode. However, this arrangement has a relatively high pressure drop due primarily to right-angle turns in the flow path, and is relatively difficult to seal properly.

### SUMMARY OF THE INVENTION

In one aspect, the present invention is directed to a porous dielectric layer disposed on a face of at least one electrode to produce a diffuse glow plasma in atmospheric pressure air. This arrangement produces a diffuse glow plasma in atmospheric pressure air while operating at a number of different operating conditions, including operating with direct current, or with alternating current at a variety of different frequencies (e.g., 60 Hz current, 20 kHz current, etc.). The dielectric material disposed on a face of at least one electrode has a tortuous pore structure that provides advantages over the linear pores or capillaries that are conventionally employed to suppress arcing in plasma generators.

Moreover, the porous electrode of the present invention produces the desired diffuse glow plasma at different operating currents (power supply frequencies) without the use of ballast resistors, which typically are employed in many types of plasma systems but represent an undesirable energy loss.

The invention also provides an improved design for a plasma generator which reduces pressure drop of a fluid passing through the plasma generator and/or provides a more homogeneous distribution of current through the space between the electrodes to produce a highly stable low-temperature, low pressure, high density plasma that is useful for sterilizing articles that are brought into contact with the plasma.

In accordance with another aspect of the invention, there is provided a plasma sterilization system comprising a plasma generator having first and second planar electrodes arranged in a spaced relationship to each other, at least one dielectric material having a tortuous pore structure disposed between the electrodes, and a plasma generating chamber defined between the electrodes. The plasma generating chamber provides an enclosure having a fluid inlet and a fluid outlet. The fluid inlet is located laterally of and between planes coinciding with major surfaces of the planar electrodes, whereby fluid entering the plasma generating chamber through the fluid inlet will flow into the plasma generating chamber between the electrodes and along a direction substantially parallel with major surfaces of the electrodes. The fluid outlet is located laterally of and between planes coinciding with major surfaces of the planar electrodes opposite the fluid inlet, whereby fluid flows into, through and out of the plasma generating chamber along a substantially straight path, and without passing through a porous or perforated material, so that there is substantially no fluid pressure drop between the fluid inlet and fluid outlet of the plasma generating chamber.

In accordance with another aspect of the invention, there is provided a process for sterilizing articles by passing an ionizable gas through the plasma generator described above, applying an ionizing electrical current to the electrodes while the ionizable gas is passing through the plasma generator, and conveying fluid from the fluid outlet of the plasma generator to and through a sterilization chamber containing an article that is to be sterilized.

These and other features, advantages and objects of the present invention will be further understood and appreciated by those skilled in the art by reference to the following specification, claims and appended drawings.

### BRIEF DESCRIPTION OF THE DRAWINGS

FIG. 1 is a schematic diagram of a plasma sterilization system.

FIG. 2 is an exploded perspective view of an exemplary plasma generator for use in accordance with the present invention.

FIG. 3 is a cross-sectional view of the plasma generator of FIG. 2 as seen along the line 3-3 of FIG. 2.

FIG. 4 is a schematic overview of a non-thermal plasma sterilization and decontamination system incorporating the electrode of FIG. 2 in accordance with the present invention.

FIG. 5 is a perspective view of a multiple unit modular sterilization section incorporating the electrode of FIG. 2 in accordance with the present invention.

FIG. 6 is a perspective view of the components of a plasma generator useful for practice of the invention and in accordance with the principles of the invention, the components being arranged in the drawing to illustrate proper assembly of the plasma generator.

### DETAILED DESCRIPTION OF PREFERRED EMBODIMENTS

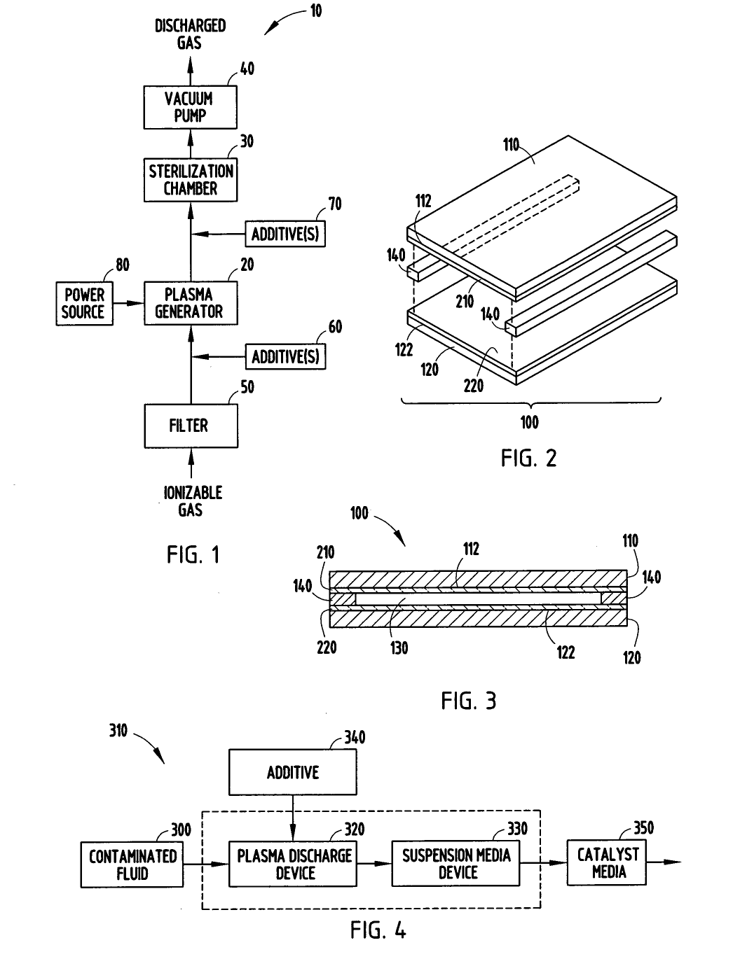

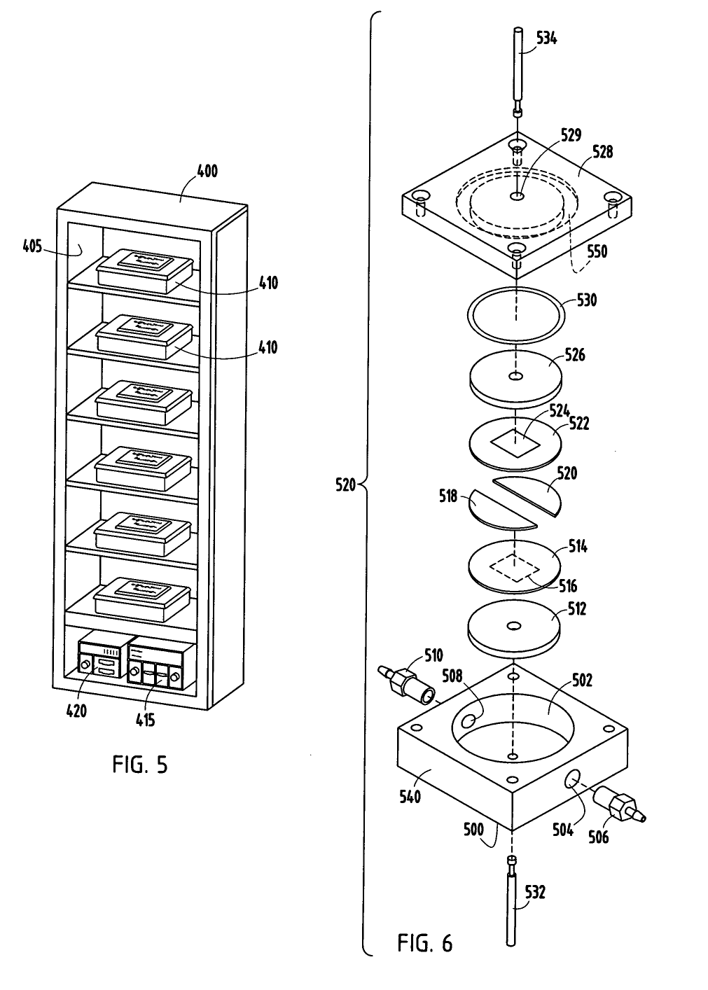

A plasma sterilization system in accordance with the invention is schematically illustrated in FIG. 1. Plasma sterilization system 10 includes a plasma generator 20 and a sterilization chamber 30. Ionizable gases flow into plasma generator 20, become partially ionized and/or form other chemically active species in the plasma generator, and the resulting plasma is discharged from plasma generator 20 into a sterilization chamber 30 which contains articles that are to be sterilized, such as medical instruments. A vacuum pump 40 is used to draw fluids through plasma generator 20 and sterilization chamber 30. It may also be desirable to draw ionizable gases such as air through a filter apparatus 50 prior to introduction into plasma generator 20 in order to remove particulate materials.

As an alternative to or in addition to vacuum pump 40, a compressor or blower may be utilized upstream of the air filter. It is also conceivable that a pressurized cylinder containing an ionizable gas could be passed through a pressure regulator, optional filter, and then into the inlet of plasma generator 20, eliminating the need for vacuum pumps and/or compressors. Desirably, for reasons of economy, simplicity and safety, the ionizable gas is ambient air. However, other ionizable gases may be employed if desired. It may also be desirable to introduce additives 60 to the ionizable gas prior to introduction of the ionizable gas into plasma generator 20 and/or to introduce additives 70 to plasma exiting plasma generator 20 before introduction into sterilization chamber 30. Examples of additives include oxidizing agents and organic additives such as alcohols and alkenes, which react with species produced in the plasma to generate highly effective sterilizing agents. In particular, it has been determined that the addition of relatively small amounts of alcohols and/or alkenes (such as ethylene) substantially reduce the time needed to achieve a desired level of sterilization. Preferably, organic additives are employed in amounts of from about 0.2% to about 2% (on a mole basis), with amounts of from about 0.5% to about 1% being preferred. In addition, it is generally beneficial to supplement ambient air with additional oxygen so that the oxygen content of the ionizable gas entering the plasma generator is above the ambient level up to about 50% on a mole basis.

An AC or DC power source 80 is electrically connected to the electrodes of plasma generator 20. While power source 80 may be an alternating current source, it is more desirable to use a direct current source to eliminate emissions of stray electromagnetic radiation which can adversely affect sensitive electronics located in close proximity to the plasma generator. It is also more desirable to use a direct current source for commercial products because DC power supplies are smaller, cheaper, and more power efficient. A suitable electrical potential across the electrodes for an AC power source is from about 2 kVᵣₘₛ to about 10 kVᵣₘₛ, and more preferably from about 3 kVᵣₘₛ to about 5 kVᵣₘₛ. A suitable electrical potential across the electrodes for a DC power source is from about 5 kVDC to about 15 kVDC. While the plasma generators of this invention have advantages relating to effective operability over a wide range of AC frequencies (e.g., from 1 kHz to 100 kHz), optimum results in terms of production of chemically active species is achieved for AC currents in the frequency range of from about 10 to about 40 kHz, preferably about 20 to about 30 kHz.

An exemplary embodiment for an electrode assembly 100 in accordance with the present invention is shown in FIGS. 2 and 3. The electrode assembly 100 includes a porous dielectric material disposed adjacent a face of at least one of the electrodes. More specifically, the electrode assembly 100 includes a first electrode (e.g., a first conductor element or plate) 110 and a second electrode (e.g., a second conductor element or plate) 120 that is spaced from the first electrode 110. As shown in FIG. 1, the first and second electrodes 110, 120 can have any number of different shapes, including a square or rectangular shape as illustrated. However, it will be appreciated that in other embodiments, the first and second electrodes 110, 120 can be formed to have other shapes, such as a circular or oval shape. In addition, the dimensions of the first and second electrodes 110, 120 can be varied and tailored to a particular application. For example, the length, width, thickness, etc., of the electrodes 110, 120 can be chosen based upon the particular application of the electrode assembly 100.

The first and second electrodes 110, 120 can be formed from any number of different materials so long as they are suitable for the intended electrode application. In other words, the first and second electrodes 110, 120 are typically formed of a conductive material, such as a metal or metal alloy, etc. Suitable materials are disclosed in the references that are expressly incorporated in the present application. Electrodes 110, 120 are preferably solid, non-porous electrodes.

According to the present invention, one or both of the first and second electrodes 110, 120 includes a layer of porous material which is generally indicated at 200 in FIG. 1. In the illustrated embodiment, the electrode 110 includes a first layer 210 of porous material that is associated with a first surface or face 112 of the first electrode 110 and a second layer 220 of porous material that is associated with a second surface or face 122 of the second electrode 120. However, it will be appreciated that in another embodiment, the porous material can be applied to only one of the first and second surfaces 112, 122. However, in direct current (DC) applications, both of the first and second electrode plates 110, 120 are covered with the first and second layers 210, 220, respectively, in order to provide a direct path to ground for the electrodes, while still maintaining the stabilizing effect provided by the porous layers 210, 220, as described below. The porous layer 210 may be fabricated of various porous ceramics, porous alumina, porous quartz, porous glass, porous plastic, etc.

Preferably, each dielectric layer 210, 220 of porous material extends across one entire surface or face, e.g., faces 112, 122, of the electrodes 110, 120, respectively. More preferably the dielectric layer(s) extend beyond the perimeter of the associated electrode(s). This construction substantially eliminates the risk of arcing across the electrodes.

In the illustrated embodiment, the face 112 of the first electrode 110 is an inner surface thereof which faces the second electrode 120 and similarly, the face 122 of the second electrode 120 is an inner surface thereof which faces the first electrode 110 such that when the two electrodes 110, 120 are spaced apart from one another, the two faces 112, 122 are opposite one another and are disposed in a space 130 that is formed between the two spaced electrodes 110, 120. It is across, the space or gap 130 that the electrons flow during normal operation of the electrode assembly 100.

In accordance with the present invention, the first and second layers 210, 220 are configured and formed from a suitable porous material to cause the first and second layers 210, 220 to act as an impediment to the flow of electrons from one electrode plate 110, 120 to the other electrode plate 120, 110. In other words, the first and second layers 210, 220 provide a flow barrier and define a tortuous path for the electrons to flow from the one electrode plate 110, 120 to the other electrode plate 120, 110 as the electrode assembly 100 is operated and the first and second electrode plates 110, 120 are hooked up to respective power supplies. The effect of providing a tortuous flow path for the electrons is that the electrodes are effectively slowed down and the plasma that is generated in the space 130 between the two electrode plates 110, 120 is stabilized.

The material that is used to form the porous layers 210,220 is selected so that it provides the desired porous characteristics and functions to define a tortuous flow path for the electrons as they flow in the space 130 from one electrode 110, 120 to the other electrode 120, 110. More specifically, the material is preferably a dielectric material that can be applied to the faces 112, 122 and provide the desired porosity that defines a tortuous flow path for the electrons as they flow from one electrode 110, 120 to the other electrode 120, 110, which leads to a stabilization of the glow discharge in the space or gap 130.

A tortuous flow path or tortuous pore structure means that the pores of the dielectric material have a network of interconnected pores that are oriented in different directions with none or substantially none of the pores extending continuously along a straight line through the dielectric material. As a result, electrons may pass through pores of the dielectric material, but must trace a non-linear path, changing direction a plurality of times in order to pass through the pores of the dielectric material. In the case of a plasma generator in accordance with the invention employing a DC power source, the tortuous pore structure must provide a pathway for electrons to pass from one side of the dielectric material to the other side of the dielectric material. However, when an AC power source is employed, the tortuous pore structure need not necessarily extend through the entire dielectric material. The pores of the porous dielectric material used for layers 210, 220 typically have diameters from the range of about 0.5 micrometers to about 20 micrometers.

The tortuous pore structure of the dielectric material allows electron energy to be dissipated more gradually and uniformly throughout the mass of the dielectric material. This eliminates or at least substantially reduces highly localized heating of the dielectric material. As a result, the possibility of thermally induced stress cracking is substantially reduced. It has also been determined that the use of a dielectric material having a tortuous pore structure facilitates generation of an atmospheric non-thermal plasma in a highly stable glow discharge regime, facilitates use of direct current, and facilitates use of a wider range of AC frequencies while suppressing glow-to-arc transition.

Atmospheric non-thermal plasma as used herein means a plasma that is generated at or near ambient pressure and temperature (e.g., within 5 psi of ambient pressure, more preferably within 3 psi of ambient pressure, and within 20° C., more preferably 10° C. of ambient temperature). The dimensions of the electrodes used in the plasma generators of this invention are not particularly critical. Various sizes and shapes for the electrodes may be employed, including circular, oval, rectangular and square. While the surface area on a face of one of the electrodes is not particularly critical, it is believed that suitable dimensions for various hospital, laboratory and commercial applications would typically range from about 0.5 square inches to about 10 square inches, and more typically from about 2 square inches to about 4 square inches. A typical spacing between either opposing dielectric layers (when each electrode has an associated dielectric layer) or a dielectric layer and an opposing electrode (when a single dielectric layer is employed) is about 0.040 inch. The dielectric layer itself is typically about 0.04 inches thick, such that the corresponding spacing between the electrodes themselves is about 0.12 inches when each electrode has an associated dielectric layer, and less than 0.1 inches when a single dielectric layer is employed. However, larger and/or narrower spacings are possible without departing from the scope and spirit of the invention, although adjustments of the power supply may be necessary or desirable depending on the spacing between the electrodes.

In one exemplary embodiment, the dielectric material is in the form of a layer of porous alumina ceramic material. However, it will be understood that this is merely one example of a suitable material that provides the desired properties described above. Other chemically similar dielectric material that are porous in nature are likewise able to be used in accordance with the present invention. For example, it will be appreciated that porous dielectric materials other than ceramics can provide the desired characteristics and serve to stabilize the glow discharge. Sintered glass and quartz materials can be used as the porous dielectric layers. Additional materials that are similar to the above materials can likewise be used to form the layers 210, 220. For example, the porous dielectric layer can be formed of a plastic material that is capable of being processed to form the desired tortuous pore structure.

As shown in the cross-sectional view of FIG. 2, the electrode assembly 100 can contain one or more spacers 140 that are provided between the two electrodes 110, 120, in the space 130, and serve to space the two electrodes 110, 120 a predetermined distance apart from one another. FIG. 2 illustrates a plurality of spacers 140 that are typically disposed at the ends (outer dimensions) of the electrodes 110, 120 and extend between the two electrodes 110, 120 such that space 130 is defined between the spacers 140. Thus, the principal area for the flow of the electrons from one electrode 110, 120 to the other electrode 120, 110 is between the spacers 140. The size and location of the spacers 140 are therefore variable and in one embodiment, the spacers 140 can be in the form of rails that extend completely across the faces 112, 122 from one edge to an opposite edge of each of the electrodes 110, 120. Alternatively, the spacers 140 can be in the form of discrete tabs or the like which are placed in select locations along the surfaces 112, 122, such as in the corners of the surfaces 112, 122 and electrodes 110, 120.

It is believed that the provision of one or more layers of dielectric material having a tortuous pore structure at a location within the gap or space 130 between the two opposing electrodes 110, 120 causes electrons to flow in a more tortuous path as the electrons flow between the two electrodes 110, 120, causing the electrons to travel at a slower speed, thereby stabilizing the plasma (glow discharge) formed between the two electrodes 110, 120.

Advantageously, the electrode assembly 100 of the present invention produces a diffuse glow plasma in atmospheric pressure air conditions. The electrode assembly 100 is capable of operating with direct current and time-varying electric fields over a wide range of operating frequencies (power supply frequencies), including frequencies of 60 Hz and 20 kHz (which are two of the more desirable power supply operating frequencies for a plasma reactor).

As described in greater detail below, the porous electrode assembly 100 is preferably used in a plasma reactor environment and therefore, in order to generate plasma, one of the electrodes 110, 120 is connected to a first terminal of a power source, while the other electrodes 120, 110 is connected to a second terminal of the power source. In this manner, a dielectric discharge can be created when the positively connected electrode 110 is positioned proximate the negatively connected electrode 120.

As previously mentioned, the present electrode assembly 100 advantageously is constructed so that the glow-to-arc transition is suppressed and instead, the electrode assembly 100 creates a “glow-like” plasma at atmospheric conditions (atmospheric pressures). More particularly, the electrode assembly 100 is constructed such that it maintains a stable, diffuse glow plasma in air at atmospheric pressure between the two electrodes. This is an improvement over the prior art electrode configurations which were limited in their success over a range of power supply frequencies.

While not generally necessary, it may be desirable in some cases to utilize auxiliary cooling (such as water-jacketing) at the electrodes of the plasma generator, and/or to provide a heat sink in thermal contact with the electrodes.

It will be appreciated that the electrode assembly 100 can be used in a variety of different plasma generation applications, including different plasma reactor schemes that utilize an electrode having the properties and capabilities of the present electrode. The following examples are merely illustrative and not limiting of the scope of the present invention, and the plasma electrode assemblies disclosed herein can be used in generally any of the plasma reactors disclosed in the patent applications/patents that have been expressly incorporated herein by reference.

### EXAMPLE 1

According to one aspect of the present invention, the plasma electrode assembly 100 is incorporated into a thermal or non-thermal plasma reactor device (plasma emitter device). FIG. 4 is an exemplary schematic flow diagram of a plasma sterilization and decontamination system in accordance with the present invention. A source of contaminated fluid 300, e.g., a liquid and/or a gas, to be treated may contain pathogens (e.g., viruses, spores) and/or undesirable chemical compounds (e.g., benzene, toluene). The contaminated fluid 300 passes through a decontamination or sterilization device 310 that includes a non-thermal plasma discharge device 320 and a suspension media 330. In accordance with the present invention, the non-thermal plasma discharge device 320 is of a dielectric plasma discharge design and utilizes an arrangement of one or more electrodes 110, 120 as illustrated in FIGS. 1-2. Although the use of a non-thermal plasma discharge device is preferred, a thermal plasma discharge device may be employed but will yield a less efficient rate of sterilization.

Energy is supplied to the non-thermal plasma discharge device 320 by a high voltage power supply, for example, a direct current, alternating current, high frequency, radio frequency, microwave, pulsed power supply, depending on the desired plasma discharge configuration. While passing through the non-thermal plasma discharge device 320, the contaminated fluid 300 is exposed to the plasma as well as to an active sterilizing species such as organic radicals and/or ion clusters created as a byproduct during the generation of the plasma. Exposure of the contaminated fluid to the plasma generated active sterilizing species substantially deactivates the pathogens and reduces concentrations of undesirable chemicals by converting them into more benign compounds.

Four reaction mechanisms that contribute to the plasma enhanced chemistry responsible for formation of the active sterilizing species will now be described. Common to all four reaction mechanisms is that of electron impact dissociation and ionization to form reactive radicals. The four reaction mechanisms include:
(1) Oxidation: e.g., conversion of CH₄ to CO₂ and H₂ O
   e⁻+0 2 →e⁻O(3P)+O(1D)
   O(3P)+CH₄→CH₃+OH
   CH₃+OH→CH₂+H₂O
   CH₂+O₂→H₂O+CO
   CO+O→CO₂
(2) Reduction: e.g., reduction of NO into N₂+O
   e⁻+N₂→e⁻+N+N
   N+NO→N₂+O
(3) Electron induced decomposition: e.g., electron attachment to CCl₄
   e⁻+CCl₄→CCl₃+Cl⁻
   CCl₃+OH→CO+Cl₂+HCl
(4) Ion induced decomposition: e.g., decomposition of methanol
   e⁻+N₂→2e⁻+N₂⁺
   N₂⁺+CH₃OH→CH₃⁺+OH+N₂
   CH₃⁺+OH→CH₂⁺+H₂O
   CH₂⁺+O₂→H₂O+CO⁺

In a preferred embodiment, an additive, e.g., an alcohol such as ethanol or methanol, may be injected into the non-thermal plasma discharge device 320 to enhance the sterilization effect or overall plasma chemistry. The additive increases the concentration of active sterilizing species generated in the plasma. Accordingly, employing an additive can advantageously be used to tailor the chemistry of the plasma generated active sterilizing species.

When organic/air mixtures are used as an additive the following chemical reaction chains are instrumental in the generation of additional active sterilizing species. Illustrative examples are provided with respect to each chemical reaction chain.
1) Formation of ions and ion clusters:
   e+N₂→N₂⁺+2 e
   e+O₂→O₂⁺+2e
   N₂⁺+N₂→N₄⁺
   O₂⁺+O₂→O₄⁺
   N₄⁺, N₂⁺+O₂→O₂⁺+products
   O₂⁺, Oₙ⁺+H₂O→O₂⁺(H₂O)
   O₂⁺(H₂O)+H₂O→O₂⁺(H₂O)₂→H₃O⁺(OH)+O₂
   H₃O⁺(OH)+H₂O→H₃O⁺(H₂O)+OH
   H₃O⁺(H₂O)+nH₂O→H₃O⁺(H₂O)₂+(n-1)H₂O→H₃O⁺(H₂O)ₕ+(n-h)H₂O

Hydronium ion clusters can protonate ethyl alcohol when present in the feed gas, as shown by the following illustrative example:
H₃O⁺(H₂O)ₕ+EtOH→EtOH₂⁺(H₂O)_{b}+(h-1-b)H₂O

Ion clusters such as EtOH₂⁺(H₂O)_{b} increase sterilization efficiency as a result of their reasonably long life time. Accordingly, ion clusters are able to survive the transport to the targeted object to be sterilized (or disinfected) and provide an Et group for replacement of a hydrogen atom in bacterial DNAs which will lead to deactivation of the targeted micro-organisms. Organic ions, such as C₂H₄OH⁺, C₂H₃OH⁺, CH₂OH⁺, CHOH⁺, CH₃OH⁺, C₂H₅⁺are also formed when an additive, free or carrier fluid is employed and may improve sterilization depending on their lifetime and chemical activity.
2) Formation of free radicals:
   e⁻+O₂→e⁻+O+O(1D)
   e⁻+O₂→e⁻+O₂*
   e⁻+N₂→+e⁻+N+N, N+O₂→NO+O
   e⁻+N₂→N₂*+e⁻, N₂*+O₂→N+O+O
   O+O₂+M→O₃+M,O₂*+O₂→O₃+O
   O(1D)+H₂O→2 OH

Other numerous chemical reactions leading to formation of NO₂, HO₂ and other active species, for example, H₂O₂, are possible.

In the presence of organics, formation of organic radicals will occur:
RH+OH→R+H₂O, R+O₂+M→RO₂+M,
RO₂+NO→RO+NO₂, RO+NO₂+M→RONO₂+M,
RO+O₂→RCHO+HO₂

Presence of organics and oxygen in plasma will also promote the formation of other organic radicals such as peroxy RO₂, alkoxy RO, acyl peroxyacyl RC(O)OO and by-products, such as hydroperoxides (ROOH), peroxynitrates (RO₂NO₂), organic nitrates (RONO₂), peroxyacids (RC(O)OOH), carboxylic acids (RC(O)OH) and peroxyacyl nitrates RC(O)O₂NO₂.

Referring once again to FIG. 3, the contaminated fluid 300 after being exposed to the generated plasma passes through a suspension media 330 (e.g., a filter, electrostatic precipitator, carbon bed or any other conventional device used to remove particulate material from fluid streams) disposed downstream of the plasma discharge device 320. Residual pathogens that have not been entirely neutralized or deactivated when exposed to the plasma discharge in the plasma discharge device are collected in the suspension media 330. These collected contaminants are treated upon contact with the suspension media 330 by the radicals and ions created by the generated plasma as part of the fluid stream. Materials, such as microorganisms that collect in the suspension media 330 react with the plasma generated active sterilizing species upon contact with the suspension media. For example, organic byproducts and radicals along with other active species interact with the DNA and other building blocks of microorganisms deposited on the suspension media device 330. By way of example, replacement of a hydrogen atom in bacterial DNA by an alkyl group (CₙH₂ₙ₊₁) due to exposure to the plasma generated active sterilizing species leads to inactivation of microorganisms. Alkylation is believed to be but one mechanism responsible for sterilization in the described method, other mechanisms and active sterilizing species may also be present.

Optionally, the plasma treated fluid may be exposed to a catalyst media 350 (e.g., an ozone catalyst) or additional suspension media disposed downstream of the suspension media 330 to further reduce concentrations of residual undesirable compounds such as ozone, pathogens, hydrocarbons, and/or carbon monoxide.

To increase concentrations of generated chemically active species, e.g., ions and free radicals, thereby accelerating and improving the overall destruction rates of undesirable chemical and/or biological contaminants, an organic based reagent may be introduced into the plasma or weakly ionized gas, as described in detail in the pending application entitled “System and Method for Injection of an Organic Based Reagent in Weakly Ionized Gas to Generate Chemically Active Species”, U.S. Patent Application Publication No. 2004/0050684, said application being incorporated by reference in its entirety. The organic based reagent may be a combination of an organic additive (e.g., an alcohol or ethylene) mixed with an oxidizer (e.g., oxygen) prior to being introduced in the weakly ionized gas. Alternatively, the organic based reagent may be the injection of an organic additive alone in the weakly ionized gas while in the presence of air (non vacuum chamber) that inherently contains oxygen and serves as the oxidizer. Also, the organic based reagent may comprise an organic additive that itself includes an oxidizing component such as ethanol. In this situation the oxidizing component of the organic component when injected into the weakly ionized gas forms hydroxyl radicals, atomic oxygen or other oxidizing species that may be sufficient to eliminate the need for a supplemental oxidizer. Regardless of the organic based reagent used, the organic additive reacts with the oxidizer while in the presence of weakly ionized gas to initiate the production of chemically active species. The modular sterilizer may be adapted to be connected to a supply source for receiving the organic based reagent into the device.

### EXAMPLE 2

In yet another embodiment and as shown in FIG. 4, one or more electrode assemblies 100 are incorporated into a system 400 for sterilizing an object, such as a piece of medical equipment, etc. For example, an arrangement of one or more electrodes 100 (FIG. 1) can be incorporated into a modular system 400 for sterilizing, disinfecting or decontamination of objects (e.g., medical instruments) utilizing non-thermal plasma and associated chemical methods, as described in U.S. Ser. No. 111042,359, filed on Jan. 24, 2005, which claims the benefit of U.S. Provisional Application No. 601538,742, filed Jan. 22, 2004. This modular sterilization system 400 can be used in other applications employing sterilization techniques such as, but not limited to, the handling of food. As disclosed in the '359 application, the disclosed modular sterilization section is configured to accommodate any number of one or more units 410 (the term “units” is generically used to describe any closable container such a tray with a lid, a closable box or a closable bag). Each unit 410 may be adapted in size and shape based on the size and shape of the particular objects being treated. The modular sterilization section is designed with one or more compartments 405 adapted in size and shape to preferably receive only one unit 410. Thus, the capacity of the modular sterilization section is limited by the number of compartments 405. By way of example, the modular sterilization section shown in FIG. 4 has six compartments 405 capable of accommodating six or less units 410, one compartment 405 being adapted to receive a single unit. A control module 415 is installed to provide electricity (either DC or AC) to and vary the parameters for each of the individual units 410. For instance, control module 415 may independently control for each unit 410 the type and quantity of an organic based reagent introduced therein, the period for sterilization, the sterilization cycles, and/or power level. It may also be desirable, but not necessary, to have the control module 415 monitor one or more parameters or conditions such as time of operation or unit status. Each unit, in turn, may be further divided or subdivided into nested compartments or sub compartments the sterilization parameters or conditions for each which again may be independently and individually controlled by the control module 415.

In a preferred embodiment, each unit 410 is adapted to produce a weakly ionized gas, e.g. plasma therein. The generation of the weakly ionized gas requires the application of an electric field to an electrode, which in this case, is preferably a configuration of electrodes 110, 120. Thus, a modular sterilization section adapted to sterilize (or disinfect) objects in situ by exposure to a gas discharge requires that each compartment 405 be electrically connected to receive energy from a power source 420 in order to generate the electric field. Correspondingly, each unit also contains electronic circuitry connected to the electrode. In a preferred embodiment, an interface or adapter, for example, complementary male and female plugs, are provided on the respective unit 410 and corresponding compartment 405 so that when the unit is inserted into a compartment the male and female connectors automatically align to complete the connection. Alternatively, cable may extend from the compartment to be manually connected to a complementary port or outlet of the unit 410.

According to one exemplary embodiment, the unit 410 can be configured as an assembled tray and complementary lid. The lid can be fabricated from a variety of materials (metallic, non- metallic, etc) and is form fit to a mating tray. A negative fit device (typically a gasket) is preferably employed to form a seal, keeping the transient biocide within the unit 410 to ensure sterility of the contents therein after the process is complete and the unit removed from the system or grid 400. A gas discharge generator for producing a weakly ionized gas is disposed to generate the transient biocide in the interior of the unit. The generator is of a dielectric discharge type as described above. Furthermore, the generator preferably incorporates the gas discharge generator in the top or lid of the unit. Positioning of the gas discharge generator may be modified so long as the weakly ionized gas is emitted into the interior of the unit with the object to be treated directly exposed to the discharge or emission.

### EXAMPLE 3

In addition, it will be appreciated that the plasma emitter device described in U.S. Pat. No. 7,098,420 can be modified so that the electrode assemblies of the present invention can be incorporated therein. More specifically, an electrode assembly in accordance with the invention can be arranged and incorporated into a plasma emitter apparatus that is portable and can be readily guided over a surface to be treated as described in the above application. The advantages of this type of plasma emitter apparatus are particularly realized with respect to a particular application of surface cleaning or treating of an object or liquid where it is desired to operate the device in air at atmospheric pressures.

Thus and according to one preferred embodiment, the electrode assembly of FIGS. 1-2 can be incorporated into plasma emitter devices that are adapted to perform sterilization and decontamination operations and enhance sterilization efficiency while reducing health and environmental hazards by employing biologically active yet relatively short living sterilizing species produced as a byproduct during the generation of non-thermal plasma, preferably in the presence of organics and oxygen.

It will be understood that the above examples are merely illustrative of potential uses for the electrodes of the present invention in a plasma generating environment and are not limiting of the scope of the present invention.

Shown in FIG. 6 are the various components of a plasma generator 520 in accordance with another embodiment of the invention. Plasma generator 520 includes a base 500 having a cylindrical recess 502. Base 500 also defines an inlet port 504 to which may be sealingly connected an inlet fitting 506, and an outlet port 508 to which may be sealingly connected an outlet fitting 510. Seated within cylindrical recess 502 is a stacked assembly comprising a lower silicone gasket 512, a bottom dielectric disk 514, a first electrode 516 attached to the underside (the side facing silicone gasket 512) of bottom disk 514, spacers 518 and 520, a top dielectric disk 522 having a second electrode 524 attached to its upper surface, and a second or upper gasket 526. Gasket 512 provides a bottom seal preventing fluids from leaking around bottom disk 514 and out of an opening provided in the bottom of cylindrical recess 502, which is provided to electrically connect a conductive pin 532 with electrode 516.

Dielectric disks 514 and 522 serve two functions. First, they are carriers for the respective electrodes 516 and 524. Second, dielectric disks 514 and 522 ensure a uniform diffuse current between the electrodes, rather than discrete arcs. Dielectric disks 514 and 522 are preferably porous when the power source connected to electrodes 516 and 524 is a time varying current. When direct current is employed, dielectric disks 514 and 522 must be porous to allow current to pass between the electrodes. However, when AC is used, dielectric plates 514 and 522 need not be porous. Further, while the illustrated embodiment includes both a bottom 514 and top dielectric disk 522, it is possible to achieve an acceptable glow discharge plasma generator using a single dielectric material between the electrodes.

Gasket 526 seals against lid 528 to prevent fluids from leaking around top disk 522 and out of an opening 529 through lid 528. Opening 529 allows a conductive pin 534 to be electrically connected with electrode 524. An O-ring 530 is compressed between an upper surface 540 of base 500 and a recess 550 in a lower surface of lid 528 to prevent fluids from leaking through interfacing surfaces of base 500 and lid 528.

Dielectric spacers 518 and 520 disposed between bottom dielectric disk 514 and top dielectric disk 522 are provided to direct substantially all fluid flow through a space bounded between planar electrodes 524 and 516. This assures that fluids passing between disks 514 and 522 flow through a space in which the electric field is strongest. This, in turn, provides an efficient arrangement in which a high plasma density is achieved (i.e., the number of chemically energetic species generated per unit volume is optimized). Spacers 518, 520 may be formed from polytetrafluoroethylene.

In the illustrated embodiment, electrodes 516 and 524 completely overlap and are of identical size. In an actual test apparatus, electrodes 516 and 524 were square metal foils having sides of 0.700 inches. The distance between dielectric disks 514 and 522 was 0.040 inches. Fluid flow rates through the plasma generator were either 0.25 liters per minute or 0.5 liters per minute. The ionizable gas introduced into the plasma generator was ambient air, optionally supplemented with oxygen to achieve an oxygen content of up to about 50 mole percent, and/or optionally supplemented with an organic additive (e.g., a C₁-C₆ alkene or alcohol) in an amount such that the organic additive would comprise from about 0.2 mole percent to about 2 mole percent of the fluid if it were not reacted by the plasma. The organic additive may be added either before the plasma generator or after the plasma generator and before the sterilization chamber. The plasma generator dimensions, fluid flow rates, etc. of the test apparatus is only illustrative of the invention, and may be varied without departing from the principles and scope of the invention.

While the plasma sterilization system of this invention is primarily intended to be used for sterilizing medical and dental instruments, it may also be used for sterilizing a variety of other articles, such as tattoo needles, baby bottles, etc.

Because fluid flows into inlet port 504 on one side of the plasma generator, laterally across the plasma generator between spacers 518 and 520, and out of outlet port 508 on the side of the plasma generator opposite the inlet port, the fluid does not make any sharp turns. As a result, there is substantially no pressure drop across the plasma generator between the inlet port and the outlet port. Consequently, it is possible to achieve higher pressure (closer to ambient) in the sterilization chamber and/or higher fluid flow rates through the sterilization chamber for a given system. Substantially no pressure drop, as used herein, typically means that the pressure drop across the plasma generator is less than 30% of the inlet pressure to the plasma generator, and more desirably less than 10%. With the illustrated arrangement utilizing a lateral flow path in which fluid flows parallel to and between the faces of the electrodes, there is typically less than a 4 psi pressure drop, and more preferably less than 2 psi pressure drop from the inlet to the outlet of the plasma generator. Stated differently, the pressure drop from the inlet of the plasma generator to the outlet of the plasma generator is typically no more than 60% of the total pressure drop between the air inlet of the sterilization apparatus to the suction side of the vacuum pump, and more desirably less than 50% of the total pressure drop. This allows relatively higher pressures and therefore relatively higher active species concentrations in the sterilization chamber. The low pressure drop across the plasma generator also allows the use of a relatively compact and inexpensive vacuum pump to achieve the desired flow through the plasma generator and sterilization chamber.

In accordance with another aspect of this invention, improved sterilization efficiency can be achieved via a pulsed flow mechanism in which the pressure in the sterilization chamber is repeatedly increased and decreased to help ensure that antimicrobially active species are contacted with all surfaces of an article that is to be sterilized in the sterilization chamber. This is particularly beneficial for sterilization of difficult to reach surfaces, such as closely spaced together surfaces and the inner walls of small diameter cannula and/or lumens. A suitable pressure cycle may have amplitude of from about 2 to about 10 psia, more typically from about 4 to about to about 7 psia, and a period (the time for a single cycle, as from one pressure minimum to the next) that is usually about 10 minutes or less, preferably 5 minutes or less, and more preferably 3 minutes or less. For many typical surgical instruments, a suitable total sterilization time, either with or without periodic pressure swings, is from about 10 to about 60 minutes, more typically from about 15 to about 45 minutes, with shorter processing times being achievable when a pulsed flow mechanism is utilized. During the pressure variations, plasma discharge is forced to flow into low pressure regions ensuring more thorough contact of difficult to reach surfaces of the article to be sterilized with the plasma discharge and therefore with antimicrobial active species in the plasma discharge. The pressure cycles may be substantially periodic or somewhat irregular. A graph of pressure in the sterilization chamber versus time may have a periodic saw-toothed shape or other wave form.

The invention in one or more of its various aspects, including the use of a dielectric material having a tortuous pore structure which is disposed on a face of at least one of the electrodes, and/or utilization of lateral flow parallel to and between the electrodes provides several advantages including the ability to produce atmospheric non-thermal plasmas from ambient air without the use of noble gases, without the use of highly sensitive ballast to inhibit discharges, and/or to operate using either an AC or DC power supply. The ability to produce antimicrobial active species at or only slightly above room temperature employing ambient air (preferably in combination with a small amount of an organic additive) is highly advantageous for sterilizing temperature sensitive articles (medical instruments and the like) in laboratories, hospitals and certain commercial applications.

All patents, patent applications, publications, procedures, and the like which are cited in this application are hereby incorporated by reference.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. An electrode arrangement for use in a plasma emitter apparatus, comprising:
a first conductor element;
a second conductor element spaced from the first conductor element such that a space is formed between opposing first faces of the first and second conductor elements;
a layer of a porous dielectric material disposed on the first face of one of the first and second conductor elements, the layer having a tortuous pore structure.

2. The electrode arrangement of claim 1, wherein the layer of porous dielectric material is disposed on the first face of the first conductor element and the first face of the second conductor element.

3. The electrode arrangement of claim 1, further including:
one or more spacers disposed between the first conductor element and the second conductor element to position the first conductor element a predetermined distance from the second conductor element.

4. A plasma reactor comprising one or more electrode arrangements of claim 1, and further including:
a DC power supply for operating the plasma reactor;
wherein a first layer of porous dielectric material is disposed on the first face of the first conductor element and a second layer of porous dielectric material is disposed on the first face of the second conductor element.

5. A method for using a plasma reactor that includes a first electrode, a second electrode spaced from the first electrode such that a space is formed between opposing first faces of the first and second electrodes, and a layer of a dielectric material having a tortuous pore structure disposed on the first face of at least one of the first and second conductor elements, said method comprising the steps of:
applying a voltage differential between the first conductor element and the second conductor element; and
generating a stable, diffuse glow plasma in the space when operating the plasma reactor in an atmospheric air pressure environment by causing electrons traveling from one of conductor element to the other to travel along a tortuous path due to the presence of pores in the layer of porous dielectric material that is disposed between the two conductor elements.

6. The method of claim 5, further including the step of:
operating the plasma reactor with direct current (DC).

7. A plasma sterilization system comprising:
a plasma generator having first and second planar electrodes arranged in a spaced relationship to each other, a layer of dielectric material having a tortuous pore structure disposed adjacent a face of at least one of the electrodes, a plasma generating chamber defined between the electrodes, the plasma generating chamber having a fluid inlet and a fluid outlet, the fluid inlet located laterally of and between planes coinciding with major surfaces of the planar electrodes, the fluid outlet located laterally of and between planes coinciding with major surfaces of the plasma electrodes at a side of the plasma generating chamber opposite the fluid inlet, whereby fluid may flow into, through and out of the plasma generating chamber along a substantially straight path; and
a sterilization chamber in fluid communication with the fluid outlet of the plasma generating chamber of the plasma generator.

8. The system of claim 7, wherein the plasma generating chamber is configured to channel substantially all fluid flow through a space bounded between the planar electrodes.

9. The system of claim 7, wherein there are two layers of dielectric material having a tortuous pore structure disposed between the electrodes, one of the layers of dielectric material disposed adjacent a major surface of a first electrode and the other layer of dielectric material disposed adjacent a major surface of the second electrode.

10. The system of claim 7, wherein the pores of the porous dielectric have diameters in the range from about 0.5 to 20 micrometers.

11. The system of claim 7, wherein the plasma generator further comprises at least one spacer disposed between the electrodes, the at least one spacer channeling fluid flow through a space bounded between the planer electrodes.

12. The system of claim 7, further comprising a vacuum pump for drawing fluid through the plasma generator and sterilization chamber.

13. A process for sterilizing articles comprising the steps of:
providing the sterilization system of claim 1;
passing an ionizable gas through the plasma generator of the sterilization system;
applying an ionizing electrical current to the electrodes while the ionizable gas is passing through the plasma generator; and
conveying fluid from the fluid outlet of the plasma generator to and through a sterilization chamber containing an article that is to be sterilized.

14. The process of claim 13, wherein a direct current is applied to the electrodes of the plasma generator.

15. The process of claim 13, wherein an alternating current is applied across the electrodes of the plasma generator at a frequency of from 1 kHz to 100 kHz.

16. The process of claim 13, wherein a pressure drop across the plasma generator is less than 30% of an inlet pressure to the plasma generator.

17. The process of claim 13, wherein the pressure in the sterilization chamber is repeatedly decreased and increased during the process, with the different between a maximum pressure and a minimum pressure in the sterilization chamber being from about 2 to about 10 psi.

18. The process of claim 17, wherein the pressure in the sterilization chamber is varied periodically, with the period from one pressure minimum to the next pressure minimum being from about 30 seconds to about 10 minutes.
